(19) United States
(12) Patent Application Publication
(10) Pub. No.: US 2025/0314317 A1
(43) Pub. Date: Oct. 9, 2025

(54) Valve, Particularly of the Type for Medical Use

(71) Applicants: Renato Sganzaroli, Corsico (IT); Massimo Sganzaroli, Rozzano (IT)

(72) Inventors: Renato Sganzaroli, Corsico (IT); Massimo Sganzaroli, Rozzano (IT)

(21) Appl. No.: 19/083,055

(22) Filed: Mar. 18, 2025

(30) Foreign Application Priority Data
Apr. 9, 2024 (EP) 24169251.6

(51) Int. Cl. F16K 11/085; A61M 39/22; F16K 31/60

(52) U.S. Cl. CPC F16K 11/0853; A61M 39/223; F16K 31/60; A61M 2039/226; A61M 2205/0222

(57) ABSTRACT

An improved valve, particularly of the type for medical use, comprising a valve body that has:
at least one first inlet;
at least one outlet;
a chamber connected to the first inlet and to the outlet;
at least one flow control element movable between at least one closed position;
a contoured protuberance defined on the bottom of the chamber;
a contoured recess defined on a first portion of the flow control element, which is designed to face toward the bottom;
the contoured protuberance and the contoured recess being geometrically shaped so as to define at least two mechanical click-stops for the relative rotation thereof which correspond, respectively, to the closed position and to the operative position.

### CROSS-REFERENCE TO RELATED APPLICATION

This application claims priority to and the benefit of European Application No. 24169251.6 filed Apr. 9, 2024, the disclosure of which is incorporated herein by this reference in its entirety.

### FIELD OF THE INVENTION

The present invention relates to an improved valve, particularly of the type for medical use.

### BACKGROUND OF THE INVENTION

Nowadays, there are many and varied types of medical valves on the market.

Although they meet current regulations, for example relating to health security, to operating tests and/or to the shape structure of some of their parts, they differ in dimensions, shapes and/or grips.

More specifically, the medical valves on the market have a plurality of inlets and outlets and a flow control element adapted to intercept and/or deviate the flow of a fluid inside one or more of such inlets and/or outlets.

In order to regulate the direction of the flow and the communication between such inlets and outlets, one particular type of conventional medical valve comprises a flow control element provided with a plurality of slots on its outer surface and proximate to/at its head, i.e. an upper portion thereof adapted to interface with a portion of the valve body, into which such flow control element is inserted, and provided with a protrusion that extends from the inner walls of the chamber.

This protrusion is contoured and adapted to be accommodated inside the cavity defined by the slots on the head of the flow control element, following a relative rotation of the latter with respect to the valve body.

Such conventional valves are not devoid of drawbacks, however.

In fact, when the relative rotation occurs between the flow control element and the valve body to pass from a first configuration of use to a second configuration of use of the valve, the protrusion on the inner walls of the chamber exerts a pressure on the head of the valve body, particularly on a portion comprised between two successive slots.

This pressure causes the head of the flow control element to deform elastically and, during the transition between two successive configurations, a proper hydraulic seal is not achieved, so leading to possible leaks of fluid.

The elastic deformation of the head owing to the contact with the protrusion generates a leak route of the fluid, which can exit from the upper part of the valve body and, if the valve body is open at the bottom, also from its lower part.

### BRIEF SUMMARY OF THE INVENTION

The aim of the present invention is to provide an improved valve, particularly of the type for medical use, that is capable of overcoming the above-mentioned drawbacks.

Within this aim, an object of the present invention consists in providing a valve that is capable of regulating the passage of one or more fluids and which is free from leaks in any condition for use of the valve.

Another object of the present invention consists in providing a valve that is easy to use and simple from the point of view of construction.

A further object of the invention is to provide a valve for which the molding process produces no waste material, for which recycling is not permitted and which therefore cannot be reused.

Another object of the present invention consists in providing a valve that is capable of offering the widest assurances of reliability, efficiency and operation.

This aim and these and other objects which will become more apparent hereinafter are achieved by an improved valve, particularly of the type for medical use.

### BRIEF DESCRIPTION OF THE DRAWINGS

Further characteristics and advantages of the invention will become more apparent from the description of two preferred, but not exclusive, embodiments of an improved valve, particularly of the type for medical use, which are illustrated by way of non-limiting example with the aid of the accompanying drawings wherein:

FIG. 1 is an exploded perspective view of a first embodiment of the valve according to the invention;

FIG. 2 is a perspective view from below of the flow control element of the valve shown in FIG. 1;

FIG. 3 is a cross-sectional view, taken along the line III-III, of the valve shown in FIG. 1;

FIG. 4 is a cross-sectional view of the valve shown in the previous figure, assembled;

FIG. 5 is a cross-sectional view of the assembled valve shown in cross-section in the previous figure, taken along the line V-V;

FIGS. 6a to 6h are transverse cross-sectional views of the first embodiment of the valve according to the invention in eight possible mutually consecutive configurations;

FIG. 7 is an exploded perspective view of a second embodiment of the valve;

FIG. 8 is a perspective view from below of the flow control element of the valve shown in FIG. 7;

FIG. 9 is a transverse cross-sectional view of the valve shown in FIG. 7, assembled, taken proximate to the bottom of the end of the flow control element;

FIGS. 10a to 10c are transverse cross-sectional views of the second embodiment of the valve according to the invention in three possible mutually consecutive configurations.

### DETAILED DESCRIPTION OF THE INVENTION

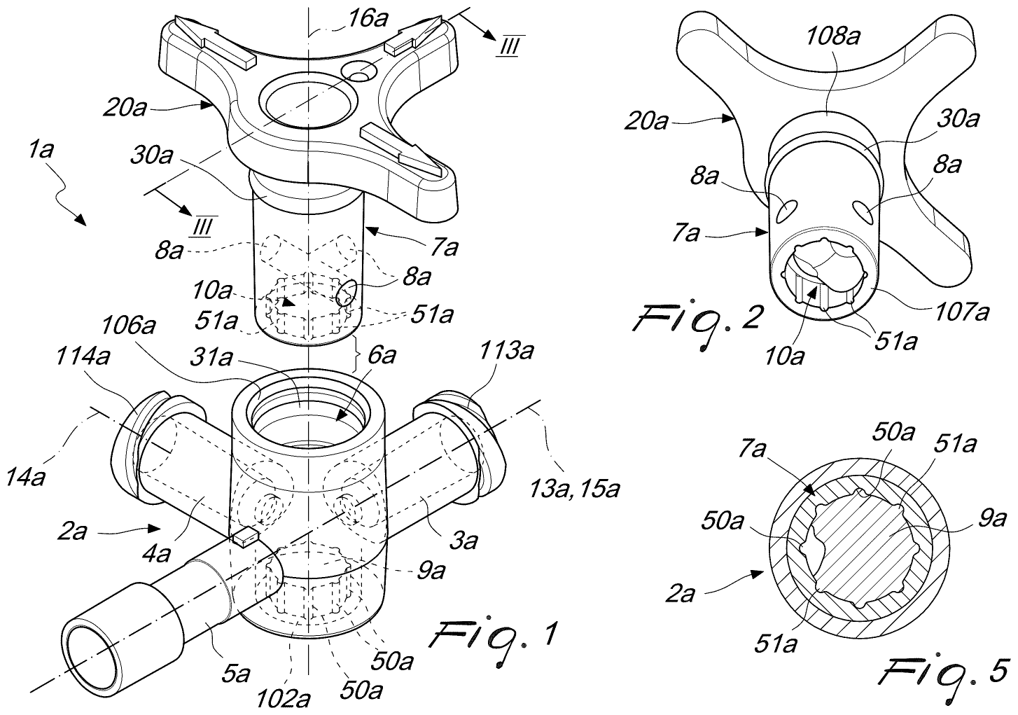

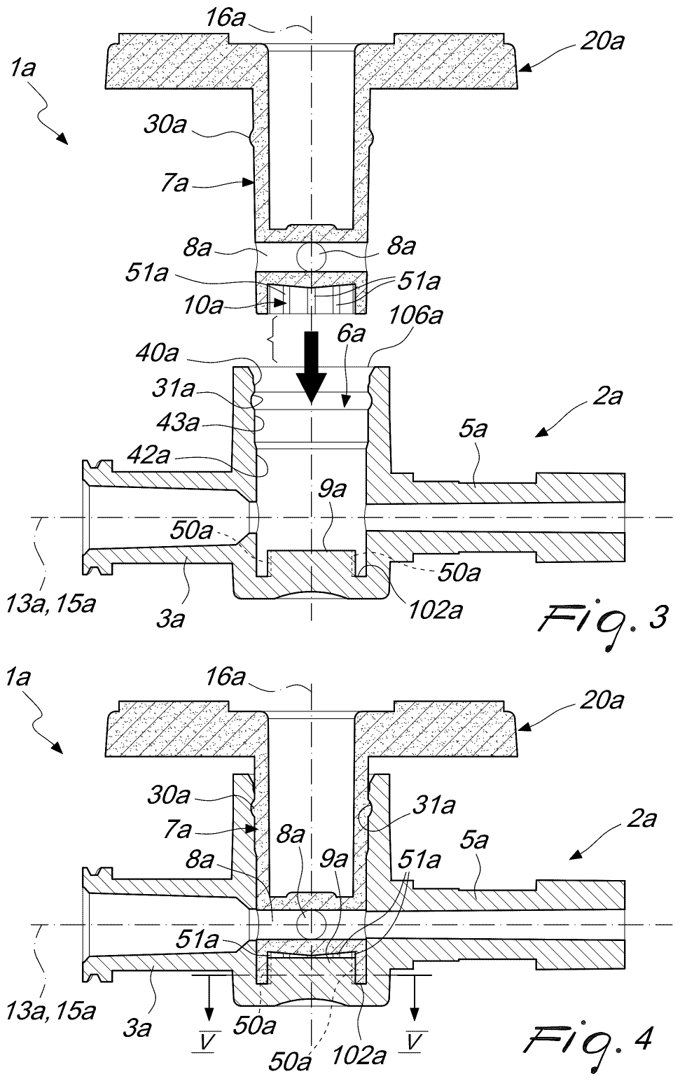

With reference to FIGS. 1 to 6h, the improved valve, particularly of the type for medical use, generally designated in the first embodiment with the reference numeral la, comprises a valve body 2a which has:
at least one first inlet 3a oriented substantially along a first axis 13a that passes through the valve body 2a and shaped to be connected to at least one first delivery pipe of a first fluid;
at least one outlet 5a oriented substantially along a second axis 15a that passes through the valve body 2a substantially incident to the first axis 13a and shaped to be connected to either at least one drainage pipe or at least one second valve;
a chamber 6a defined at the intersection of the axes 13a and 15a in such a manner as to be passed through by them and be connected to the first inlet 3a and with the outlet 5a.

At least one flow control element 7a is further comprised, which is accommodated so that it can rotate inside the chamber 6a and has, on its outer surface, a plurality of ports 8a which are mutually connected.

The flow control element 7a is movable between at least one closed position, in which the chamber 6a is hydraulically isolated from either or both of the first inlet 3a and the outlet 5a, and at least one operative position, in which the first inlet 3a and the outlet 5a are mutually connected through the ports 8a.

According to the invention, the valve la comprises:
a contoured protuberance 9a defined on either the bottom 102a of the chamber 6a or a first portion 107a of the flow control element 7a, which is designed to face toward such bottom 102a;
a contoured recess 10a defined on the other one of either the bottom 102a or the first portion 107a.

Advantageously, the contoured protuberance 9a and the contoured recess 10a are geometrically shaped so as to define at least two mechanical click-stops for the relative rotation of the flow control element 7a with respect to the chamber 6a which correspond, respectively, to the closed position and to the operative position.

The expression “mechanical click-stops” means relative positions between the flow control element 7a and the chamber 6a that are stable and which define at least two configurations of use of the valve la.

As will be better described later, in this first embodiment, the contoured protuberance 9a is defined on the bottom of the chamber 6a, while the contoured recess 10a is defined on the first portion 107a of outer surface of the flow control element 7a which is designed to face toward the bottom 102a of the chamber 6a.

Advantageously, the valve la comprises activator means 20a of the flow control element 7a, which are directly associated therewith at a second portion 108a thereof that is external with respect to the chamber 6a and arranged opposite with respect to the first portion 107a.

Advantageously, such activator means 108a comprise a control knob with an ergonomic shape that can be manipulated manually by an operator, for example, by means of the use of two fingers.

Conveniently, the chamber 6a is substantially frustum-shaped with a blind circular base, extending along a third axis 16a which is incident to the first axis 13a and to the second axis 15a.

More specifically, the bottom 102a of the chamber 6a is defined at the minor base of the substantial frustum shape with a blind circular base.

Such a shape of the chamber 6a makes it possible to manufacture the valve body 2a using the direct injection technique, a process that produces substantially no waste material.

Advantageously, the flow control element 7a has a geometry that is substantially complementary to the substantial frustum shape with a blind circular base of the chamber 6a, so as to be accommodated inside such chamber 6a and creating a hydraulic seal with its walls.

In fact, as will be better described later, the flow control element 7a and the chamber 6a have particular geometric contrivances that create a hydraulic seal between them.

Conveniently, furthermore, the valve la comprises extraction-preventing means 30a and 31a of the flow control element 7a with respect to the chamber 6a along the third axis 16a.

In fact, the chamber 6a has an aperture 106a arranged opposite to the bottom 102a, for inserting the flow control element 7a into the chamber 6a along the third axis 16a.

Such extraction-preventing means 30a and 31a are defined partially by the flow control element 7a and partially by the chamber 6a.

Advantageously, such extraction-preventing means 30a and 31a comprise at least one annular ridge 30a defined on either the inner surface of the chamber 6a or the outer surface of the flow control element 7a, on a plane perpendicular to the third axis 16a, and a respective annular seat 31a defined on the other one of either the inner surface of the chamber 6a or the outer surface of the flow control element 7a.

The annular ridge 30a has a blended outer surface in order to allow the forced insertion of the flow control element 7a into the chamber 6a.

In the specific case of the first embodiment described here, the annular ridge 30a extends circumferentially on the outer surface of the flow control element 7a, on a plane perpendicular to the third axis 16a.

More specifically, such annular ridge 30a is defined, along the third axis 16a, on a portion of outer surface of the flow control element 7a comprised between the second portion 108a and the part of the chamber 6a that is connected to the first inlet 3a and the outlet 5a.

The annular seat 31a by contrast is defined on the inner walls of the chamber 6a, and has a geometry substantially complementary to the geometry that defines the annular ridge 30a.

In this manner, as a result of the insertion of the flow control element 7a into the chamber 6a, an extraction-preventing joint is created that makes it possible to manipulate the valve la without the risk of the flow control element 7a slipping out and exiting the chamber 6a in which it is accommodated.

Advantageously, the chamber 6a comprises a convergent section 40a that extends along the third axis 16a from the aperture 106a to the extraction-preventing means 30a and 31a, and the aperture 106a has an inner edge that is beveled for the facilitated insertion of the flow control element 7a into the chamber 6a.

Such chamber 6a further comprises an end section 42a, which extends at the first inlet 3a and at the outlet 5a and is radially narrower than the convergent section 40a and shaped so as to allow the forced accommodation of part of the flow control element 7a.

In this way a hydraulic seal is created between the inner walls of the chamber 6a and the outer walls of the flow control element 7a.

Furthermore, more specifically, between the sections 40a and 42a described above a straight section 43a is provided which is radially widened with respect to the end section 42a and is adapted to define a variation in transverse cross-section that is such as to contribute to the hydraulic seal between the chamber 6a and the flow control element 7a.

Advantageously, the valve la further comprises at least one second inlet 4a, oriented substantially along a fourth axis 14a that passes through the valve body 2a substantially incident to the first axis 13a, to the second axis 15a and to the third axis 16a, and which is adapted to be connected to a second delivery pipe of a second fluid.

Conveniently, the first axis 13a and the second axis 15a are substantially coincident.

Furthermore, the fourth axis 14a is substantially perpendicular to the first axis 13a and to the second axis 15a, and the third axis 16a is substantially perpendicular to the plane defined by the first axis 13a, by the second axis 15a and by the fourth axis 14a.

The number of ports 8a in the flow control element 7a is conveniently equal to the overall number of inlets and outlets on the valve body 2a; therefore, in the case considered here, the flow control element 7a has three ports 8a, respectively a first, a second and a third port.

The three ports 8a extend radially, substantially from the outer surface of the flow control element 7 with respect to the third axis 16a, where they are substantially incident.

The first port and the second port are substantially coaxial and are oriented along two parallel and coincident axes that substantially intersect the third axis 16a.

The third port is instead oriented along an axis that is substantially perpendicular, both to the two parallel and coincident axes and also to the third axis 16a or 16b.

The three ports extend substantially on a plane perpendicular to the third axis 16a, which substantially coincides with the plane that contains the first axis 13a, the second axis 15a and the fourth axis 14a.

In this manner, the axes on which the three ports are oriented are alignable respectively with the first axis 13a, the second axis 15a and the fourth axis 14a, following a relative rotation between the flow control element 7a and the chamber 6a.

The control knob is shaped like a “T” and has a substantially flat extension on a plane substantially perpendicular to the third axis 16a, reproducing the inner arrangement of channels of the ports 8a.

Furthermore, the first inlet 3a and the second inlet 4a respectively have a first threaded connector 113a and a second threaded connector 114a, which are adapted to be engaged respectively by a first threaded coupling and by a second threaded coupling, at the end of the first and/or of the second delivery pipe.

The outlet 5a on the other hand comprises a third connector 115a with a substantially frustum-shaped geometry adapted to be engaged by a third coupling at the end of the drainage pipe or of the second valve.

More specifically, such third connector 115a is a Luer cone.

Advantageously, either or both of the flow control element 7a and the valve body 2a is made of a softer and more elastically yielding material than the material with which the other one of either the flow control element 7a or the valve body 2a is made.

More specifically, the valve body 2a is made of polycarbonate and the flow control element 7a is made of polyolefin material with a lower hardness than polycarbonate, for example high-density polyethylene (HDPE).

Conveniently, furthermore, the valve la comprises a layer of silicone material interposed between the valve body 2a and the flow control element 7a, which is adapted to lubricate the flow control element 7a and to allow its relative rotation with respect to the valve body 6a.

The layer of silicone material is deposited, at the discretion of the assembly technician, on the inner surface of the chamber 6a and/or on the outer surface of the flow control element 7a, before the latter is inserted into the chamber 6a.

With reference to the first embodiment of the valve la, the geometry of the contoured protuberance 9a advantageously is substantially circular around the third axis 16a, with at least two semi-cylindrical lateral protuberances 50a aligned parallel with the third axis 16a.

Conveniently, the geometry of the contoured recess 10a is complementary to the contoured protuberance 9a, with at least two lateral seats 51a which correspond to such at least two lateral protuberances 50a.

Furthermore, the coupling of such at least two lateral protuberances 50a with such at least two lateral seats 51a defines the above-mentioned at least two mechanical click-stops.

In this, not least by virtue of the chosen materials, the two mechanical click-stops are elastically yielding so as to permit forced relative rotations of the contoured protuberance 9a in the contoured recess 10a and, at the same time, the exact positioning of the flow control element 7a in at least one from among such at least one closed position and such at least one operative position, as a result of the elastic yielding of either or both of the contoured protuberance 9a and the contoured recess 10a.

Advantageously, a plurality of lateral protuberances 50a is provided around the contoured protuberance 9a according to a circular array at the same constant pitch, so as to define a plurality of mechanical click-stops with constant pitch.

Furthermore, such at least one operative position therefore comprises a plurality of operative positions which correspond to at least some of the plurality of mechanical click-stops with constant pitch.

More specifically, the second lateral protuberances 50a extend on the outer side of the contoured protuberance 9a according to a semi-cylindrical geometric shape with an axis of longitudinal extension substantially parallel to the third axis 16a.

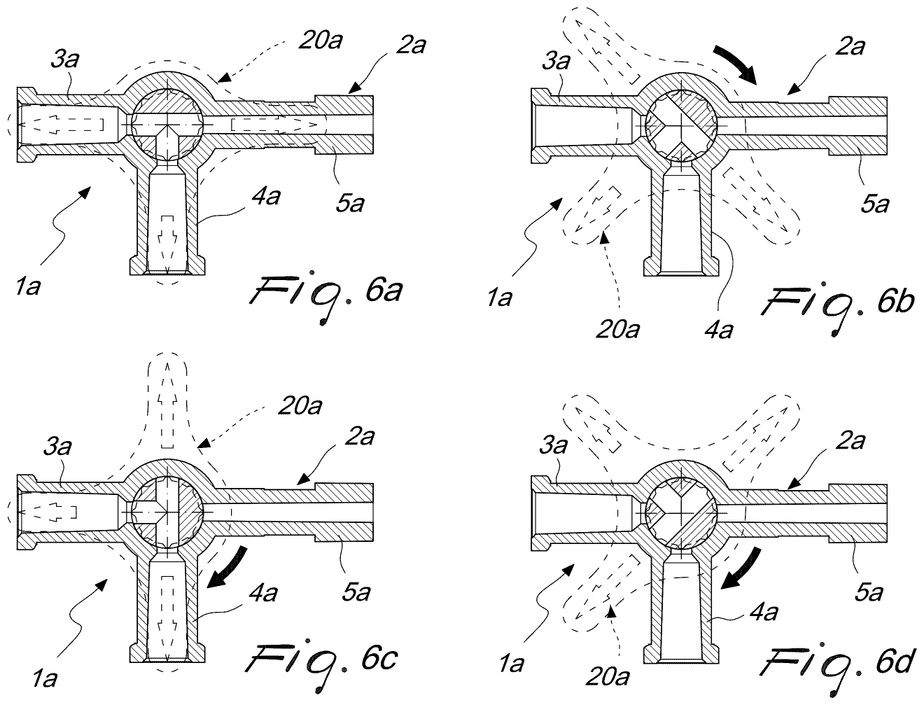

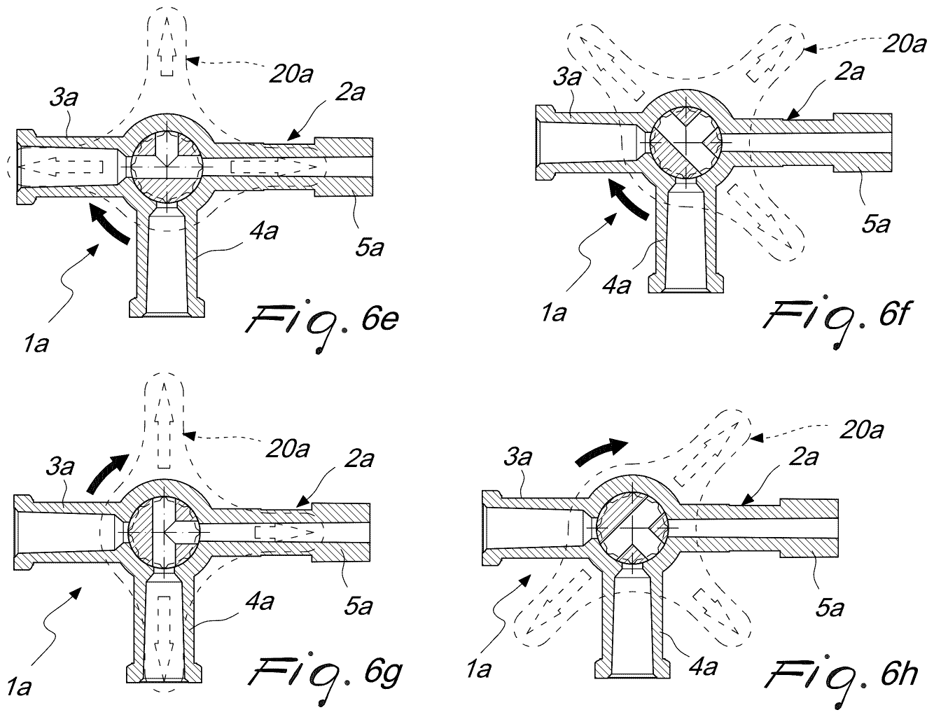

In the first embodiment, also, the contoured protuberance 9a has eight lateral protuberances 50a and the contoured recess 10a has eight corresponding lateral seats 51a, so as to define eight relative positions between the flow control element 7a and the valve body 2a, each one defined by a partial rotation of the flow control element 7a substantially equal to 45°, as shown in FIGS. 6a to 6h.

Advantageously, the plurality of operative positions comprises:
a first operative position, in which the first inlet 3a, the second inlet 4a and the outlet 5a are hydraulically connected to the chamber 6a, as shown in FIG. 6a;
a second operative position, in which the first inlet 3a and the outlet 5a are hydraulically connected to the chamber 6a, while the second inlet 4a is hydraulically isolated, as shown in FIG. 6e; and
a third operative position, in which the second inlet 4a and the outlet 5a are hydraulically connected to the chamber 6a, while the first inlet 3a is hydraulically isolated, as shown in FIG. 6g.

Conveniently, furthermore, such at least one closed position comprises:
a first closed position, in which the first inlet 3a and the second inlet 4a are hydraulically connected to the chamber 6a, while the outlet 5a is hydraulically isolated, as shown in FIG. 6c; and
a plurality of second closed positions, corresponding to intermediate positions between such operative positions and such first closed position, in which the first inlet 3a, the second inlet 4a and the outlet 5a are hydraulically isolated, as shown respectively in FIGS. 6b, 6d, 6f and 6h.

In this way, the valve la can be used as a three-way valve.

Differently, if one of the inlets 3a and 4a should be used as an outlet, even temporarily, for the flow of fluid originating from the inlet, then the “first closed position” would take on the functionality of a “fourth operative position”.

In this way, the valve la can be used as a four-way valve.

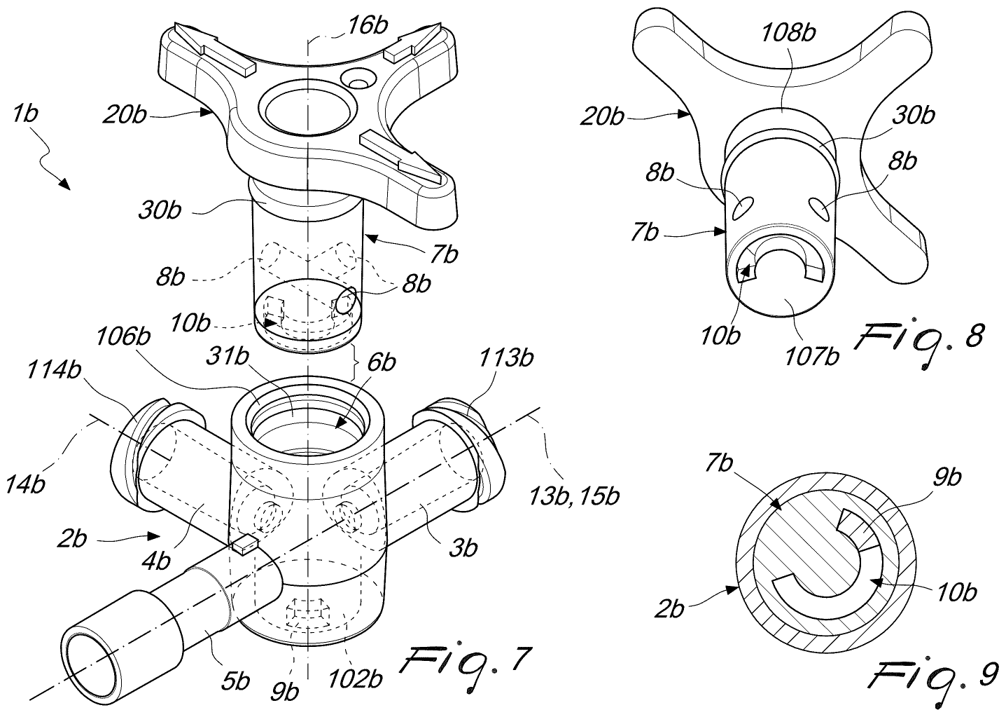

With reference to FIGS. 7 to 10c, the improved valve, particularly of the type for medical use, is generally designated in the second embodiment with the reference numeral 1b.

Such second embodiment has parts, elements and characteristics in common with or analogous to the first embodiment described above.

For this reason, in the figures cited above, these common or analogous parts, elements and characteristics, if shown, are indicated with the same reference numbers used for the first embodiment, replacing the subscript “a” with the subscript “b”.

Differently from the first embodiment, the contoured recess 10b instead has a geometric shape that is substantially an arc of an annulus and the contoured protuberance 9b is accommodated therein with play so that it allows relative rotations between the flow control element 7b and the chamber 6b that are equal to the difference between the extent of the arc of an annulus and the thickness of the contoured protuberance 9b along the average circumference of said arc of an annulus.

More specifically, in the valve 1b shown in FIG. 7, the geometry of the contoured protuberance 9b is also that of an arc of an annulus, of lesser extent than the extent of the arc of an annulus that defines the geometric shape of the recess 10b.

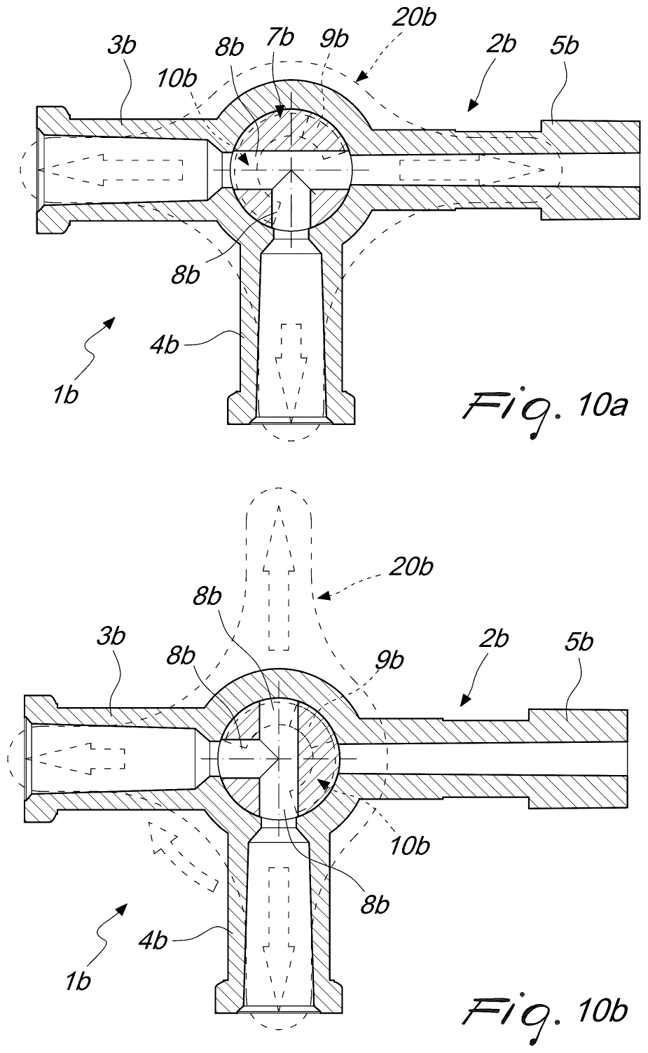

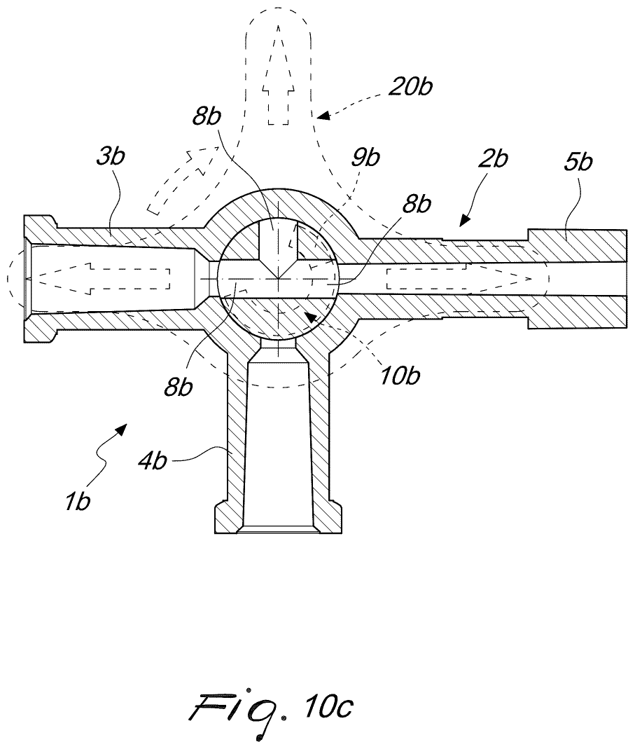

In this way, it is possible to select the degrees of rotation of the flow control element 7b, so as to have a rotation limited to 90°, as shown in FIGS. 10a to 10b, so providing a two-way valve, or a rotation limited to 180°, as shown in FIGS. 10a to 10c, so providing a three-way valve.

The operation of the valve 1a or 1b is clear and easily intuited from the foregoing description.

More specifically, relative rotations between the flow control element 7a or 7b and the valve body 2a or 2b make it possible to define different configurations of use of the valve la or 1b, which permit and/or stop the passage of a fluid through the inlets and/or the outlet of the valve body 2a or 2b, so regulating the direction of its flow.

In practice it has been found that the improved valve, particularly of the type for medical use, according to the present invention achieves the set aim and objects, in that it makes it possible to regulate the passage of one or more fluids while preventing leaks in any configuration of use, by virtue of the continuity of the fluid-tight walls between the flow control element and the internal chamber.

In fact, the particular shape of the bottom of the chamber and of the flow control element means the above-mentioned fluid-tight walls are not subject to deformations during the rotation of the flow control element inside the chamber.

Another advantage of the valve according to the present invention consists in that it can be easily used and it is simple in terms of construction.

Another advantage of the valve according to the invention consists in that it is made with mechanical machining processes that substantially do not produce waste material.

Another advantage of the valve according to the invention consists in that, by virtue of the chamber with a closed bottom, it further limits the possibility of leaks.

The valve, thus conceived, is susceptible of numerous modifications and variations, all of which are within the scope of the appended claims.

For example, the inlets and the outlet described here can have different shape structures according to the coupling required with the delivery and/or drainage pipes.

Furthermore, the functionality of said inlets/outlet can be inverted, and the latter can act respectively as outlets/inlet according to requirements.

Moreover, all the details may be substituted by other, technically equivalent elements.

In practice the materials employed, provided they are compatible with the specific use, and the contingent dimensions and shapes, may be any according to requirements and to the state of the art.

What is claimed is:

1. An improved valve, particularly of a type for medical use, comprising a valve body that has:
at least one first inlet oriented substantially along a first axis that passes through said valve body and shaped to be connected to at least one first delivery pipe of a first fluid;
at least one outlet oriented substantially along a second axis that passes through said valve body substantially incident to said first axis and shaped to be connected to either at least one drainage pipe or at least one second valve;
a chamber defined at an intersection of said first and second axes in such a manner as to be passed through by said first and second axes and be connected to said at least one first inlet and with said at least one outlet;
at least one flow control element being further comprised which is accommodated so that it can rotate inside said chamber and has, on an outer surface thereof, a plurality of ports which are mutually connected;
said at least one flow control element being movable between at least one closed position, in which said chamber is hydraulically isolated from either or both of said at least one first inlet and said at least one outlet, and at least one operative position, in which said at least one first inlet and said at least one outlet are mutually connected through said ports;
further comprising:
a contoured protuberance defined on either a bottom of said chamber or a first portion of said flow control element, which is designed to face toward said bottom;
a contoured recess defined on another one of either said bottom or said first portion;
said contoured protuberance and said contoured recess being geometrically shaped so as to define at least two mechanical click-stops for a relative rotation of said flow control element with respect to said chamber which correspond, respectively, to said at least one closed position and to said at least one operative position.

2. The valve according to claim 1, wherein said valve comprises activator means of said flow control element which are directly associated with said flow control element at a second portion of said flow control element that is external with respect to said chamber and arranged opposite with respect to said first portion.

3. The valve according to claim 1, wherein said chamber is substantially frustum-shaped with a blind circular base extending along a third axis which is incident to said first axis and said second axis; the bottom of said chamber being defined at a minor base of said substantial frustum shape with a blind circular base.

4. The valve according to claim 3, wherein said flow control element has a geometry substantially complementary to said substantial frustum shape with a blind circular base of said chamber.

5. The valve according to claim 3, further comprising extraction-preventing means of said flow control element with respect to said chamber along said third axis and defined partially by said flow control element and partially by said chamber; said chamber having an aperture arranged opposite to said bottom for insertion of said flow control element into said chamber along said third axis.

6. The valve according to claim 5, wherein said extraction-preventing means comprise at least one annular ridge defined on either the inner surface of said chamber or the outer surface of said flow control element, on a plane perpendicular to said third axis, and a respective annular seat defined on the other one of either said inner surface of said chamber or said outer surface of said flow control element; said annular ridge having a blended surface in order to allow a forced insertion of said flow control element into said camera.

7. The valve according to claim 6, wherein said chamber comprises a convergent section, extending along said third axis from said aperture to said extraction-preventing means and wherein said aperture has an inner edge that is beveled for the facilitated insertion of said flow control element into said chamber; said chamber further comprises an end section, which extends at said at least one first inlet and at said at least one outlet and is radially narrower than said convergent section and shaped so as to allow the forced accommodation of part of said flow control element.

8. The valve according to claim 3, further comprising at least one second inlet, oriented substantially along a fourth axis that passes through said valve body substantially incident to said first axis, to said second axis and to said third axis, and is adapted to be connected to a second delivery pipe of a second fluid.

9. The valve according to claim 8, wherein:
said first axis and said second axis are substantially coincident,
said fourth axis is substantially at right angles to said first axis and to said second axis and
said third axis is substantially perpendicular to a plane defined by said first axis, said second axis and said fourth axis.

10. The valve according to claim 1, wherein either or both of said flow control element and said valve body is made of a softer and more elastically yielding material than a material with which the other one of either said flow control element or said valve body is made.

11. The valve according to claim 1, further comprising a layer of silicone material interposed between said valve body and said flow control element, which is adapted to lubricate said flow control element and is adapted to allow a relative rotation of said flow control element with respect to said valve body.

12. The valve according to claim 3, wherein said contoured protuberance has a substantially circular geometry around said third axis with at least two semi-cylindrical lateral protuberances aligned parallel with said third axis and wherein the geometry of said contoured recess is complementary to said contoured protuberance with at least two lateral seats which correspond to said at least two lateral protuberances; the coupling of said at least two lateral protuberances with said at least two lateral seats defines said at least two mechanical click-stops.

13. The valve according to claim 12, wherein said at least two lateral protuberances comprise a plurality of lateral protuberances arranged around said contoured protuberance in a circular array with constant pitch so as to define a plurality of mechanical click-stops with constant pitch; said at least one operative position comprising a plurality of operative positions of said flow control element which correspond to at least part of said plurality of mechanical click-stops with constant pitch.

14. The valve according to claim 13, wherein said plurality of operative positions comprise:
a first operative position, in which said at least one first inlet, said at least one second inlet and said at least one outlet are hydraulically connected to said chamber;
a second operative position, in which said at least one first inlet and said at least one outlet are hydraulically connected to said chamber, while said at least one second inlet is hydraulically isolated; and
a third operative position, in which said at least one second inlet and said at least one outlet are hydraulically connected to said chamber, while said at least one first inlet is hydraulically isolated;
and wherein said at least one closed position comprises:
a first closed position, in which said at least one first inlet and said at least one second inlet are hydraulically connected to said chamber, while said at least one outlet is hydraulically isolated; and
a plurality of second closed positions, corresponding to intermediate positions between said operative positions and said first closed position, in which said at least one first inlet, said at least one second inlet and at least one outlet are hydraulically isolated.

15. The valve according to claim 1, wherein said contoured recess has a geometric shape that is substantially an arc of an annulus and wherein said contoured protuberance is accommodated with play in said contoured recess so that relative rotations between said flow control element and said chamber are allowed that are equal to a difference between an extent of said arc of an annulus and a thickness of said contoured protuberance along an average circumference of said arc of an annulus.
